(19) United States
(12) Patent Application Publication
(10) Pub. No.: US 2023/0000322 A1
(43) Pub. Date: Jan. 5, 2023

(54) PROBE

(71) Applicant: ODI MEDICAL AS, Oslo (NO)

(72) Inventors: Svein-Erik Måsøy, Trondheim (NO); Anastasiya Dykyy, Oslo (NO); Jochen Rau, Trondheim (NO); Øyvind Standal, Trondheim (NO); Ute Wiesemann, Freiburg (DE)

(21) Appl. No.: 17/772,040

(22) PCT Filed: Nov. 2, 2020
(86) PCT No.: PCT/EP2020/080710

§ 371 Date: Sep. 9, 2022

(30) Foreign Application Priority Data
Oct. 31, 2019 (GB) 1915837.7

(51) Int. Cl. A61B 1/00; A61B 5/00; A61B 5/02

(52) U.S. Cl. CPC A61B 1/00137; A61B 5/0075; A61B 5/02028

(57) ABSTRACT

A system for measuring haemodynamic parameters relating to a subject, the system comprising a DRS probe, a microscope probe, and a cap for use at a respective distal end of each of the probes, one at a time. The cap comprises a rigid flat contact surface for contact with a body surface of a subject, the contact surface comprising an aperture arranged such that, in use, the aperture is optically aligned with the optical probe. A rigid side wall surrounds the contact surface, defining a closed end and an open end, the closed end being formed by the contact surface. The open end is arranged to receive the probe in use. The side wall is arranged such that, in use, the cap is held in abutment against the probe. A securing portion is arranged to removably secure the cap to the probe.

### TECHNICAL FIELD

The present invention relates to caps for use with diagnostic probes, for example with diffuse reflectance spectroscopy and microscopy probes.

### BACKGROUND ART

Acute cardiac heart failure compromises central haemodynamics and consequently microvascular perfusion throughout the body. The mortality rate varies from 50 to 80 percent. Veno-arterial extra-corporeal membrane oxygenation (va-ECMO) may be used as a bridge to recovery or to other destination therapy. However, only approximately one-third of adult patients treated on ECMO for cardiogenic shock survive. ECMO treatment is resource-demanding and the assumption underlying its use, that improved technological solutions and haemodynamics—i.e. improved blood pressure and cardiac output—translates into improved survival, may not be completely valid.

As outlined in the Applicant's earlier publication WO 2014/114814, as well as focussing on central haemodynamics, it is important to characterise the state/quality of the patient's microcirculation, i.e. to collect parameters related to oxygen delivery to the cells. The Applicant has previously outlined methods for collecting these parameters, e.g. through the use of diffuse reflectance spectroscopy (DRS) and microscopy, including video microscopy.

Those skilled in the art will appreciate that DRS utilises visible and near-infrared (NIR) light in order to determine the concentration of haemoglobin in a patient's blood. DRS also provides information regarding the oxygenation of the patient's blood. The patient's skin is illuminated with relatively broadband light using optical fibre probes, and the light reflected by superficial capillaries in the patient's skin is used to determine the haemoglobin concentration and blood oxygenation.

In an exemplary method, a number of DRS recordings (e.g. twelve) are captured from a skin area of roughly 1-2 cm². The DRS probe is relocated for each of the recordings in order to provide statistical data for averaging and calculation of coefficient of variation. From this recording, the level of local oxygenation in the skin capillaries may be extracted by a suitable algorithm.

A number of videos (e.g. five) may be recorded using the microscope capturing a video sequence (e.g. of 20 second duration) for each of the recordings. As with the DRS procedure, the microscope is relocated for each recording, where the total area covered should roughly be the same as for the DRS procedure. From these videos, the capillary density (i.e. the capillaries per square millimeter) are counted by human visual analysis and the velocities are extracted, typically by a human grading against a scale, e.g. ranging from 0 to 5. This process may also be carried out by a computer or computerised system.

When the microscope is used, baby oil (or a similar type of oil), is used on the skin surface to avoid specular reflections from the skin surface. Generally, this is why the spectroscopy recording is carried out first, as this does not need oil.

The Applicant has appreciated, however, that the measurements taken can be particularly sensitive to the angle of incidence, i.e. the angle between the probe and the patient's skin. Spectroscopy results are particularly sensitive to angle variations, where large angles may result in specular reflections, affecting the shape of the spectra. In the worst-case scenario, this may again affect oxygenation data by providing a large variation in the data due to different angles while recording.

The impact of tilt on measurements can be understood with reference to FIG. 9, which is a chart that shows the O₂ estimate for varying degree of probe tilt. The chart of FIG. 9 is split into nine cells, numbered 1-9. Each cell contains six different measurements (the different colouration representing oxygenation level) using the angle as described on the x- and y-axis (no tilt, some tilt, and severe tilt). The x-axis represents variations in angle measured on the skin surface, while the y-axis represents variations in angle on the white reference.

For each measurement on the skin, there is a corresponding measurement on a white reference. In use, the skin measurements may be divided by the corresponding white reference measurements for calibration. For example in cell 7, both the skin and white reference measurements are recorded with no tilt/angle on the spectroscopy probe (i.e. normal to the surface). In cell 1, the white reference measurement is recorded with a severe angle, but the skin is recorded with no tilt. In cell 3, the measurements on both skin and the white reference have a severe angle. It can be seen from FIG. 9 that the angle on the white reference does not matter for the oxygen estimates on skin when there is no angle on the skin surface. However, as the angle towards the skin changes from no angle to severe, the degree of tilt has a significant impact on the estimated oxygenation, independent of the angle towards the white reference.

The Applicant has appreciated the important of avoiding any tilt at all, e.g. by always having a normal angle towards any measurement surface (such as a subject's skin), so as to avoid these issues which are at least partly caused by specular reflections from the skin which increase with angle.

Thus the present invention seeks to provide a mechanism to improve the repeatability and robustness of such measurements.

### SUMMARY OF THE INVENTION

In accordance with a first aspect, the present invention provides a system for measuring haemodynamic parameters relating to a subject, the system comprising:
a DRS probe;
a microscope probe; and
a cap for use at a respective distal end of each of the probes, one at a time, wherein the cap comprises:
a rigid flat contact surface for contact with a body surface of a subject, said contact surface comprising an aperture, said aperture being arranged such that, in use, the aperture is optically aligned with the optical probe in use;
a rigid side wall surrounding the contact surface, wherein the side wall defines a closed end and an open end, said closed end being formed by the contact surface and said open end being arranged to receive the probe in use, said side wall being arranged such that, in use, the cap is held in abutment against the probe; and
a securing portion arranged to removably secure the cap to each of the probes.

This first aspect of the invention extends to an optical probe arrangement for measuring haemodynamic parameters relating to a subject, the system comprising:
an optical probe; and
a cap for use at said distal end of the probe, wherein the cap comprises:
a rigid flat contact surface for contact with a body surface of a subject, said contact surface comprising an aperture, said aperture being arranged such that, in use, the aperture is optically aligned with the optical probe;
a rigid side wall surrounding the contact surface, wherein the side wall defines a closed end and an open end, said closed end being formed by the contact surface and said open end being arranged to receive the probe, said side wall being arranged such that, in use, the cap is held in abutment against the probe; and
a securing portion arranged to removably secure the cap to the probe.

This first aspect of the invention further extends to a cap for use at a distal end of an optical probe, wherein the cap comprises:
a rigid flat contact surface for contact with a body surface of a subject, said contact surface comprising an aperture, said aperture being arranged such that, in use, the aperture is optically aligned with the optical probe;
a rigid side wall surrounding the contact surface, wherein the side wall defines a closed end and an open end, said closed end being formed by the contact surface and said open end being arranged to receive the probe, said side wall being arranged such that, in use, the cap is held in abutment against the probe; and
a securing portion arranged to removably secure the cap to the probe.

Thus it will be appreciated that aspects of the present invention provide a protective cap that may be positioned at the end of a probe that reduces, and potentially eliminates, deviations in the angle of incidence between the probe and the body surface of the subject (e.g. the subject's skin). It will be appreciated by those skilled in the art that the term ‘rigid’ as used herein means that, in standard use, the material does not deform or bend out of shape, i.e. it is not flexible. Thus when an operator moves the probe on the body surface using pressure typical for such a procedure, the cap remains in shape and does not flex, thereby reducing the ability for the angle of incidence between the probe and the body surface to change.

Additionally, the flat contact surface of the cap may advantageously reduce the pressure on the subject's body surface. While an operator may generally be trained to apply as light a pressure as possible for the subject's comfort, the Applicant has appreciated that due to the relatively small surface area of some probes, it is possible that the use of conventional probes may impede a subject's blood flow during examination. A cap in accordance with embodiments of the present invention may help to prevent such a reduction in the subject's blood flow by ‘spreading’ the force of the probe across a greater area of the body surface. The degree to which the pressure is spread out will, of course, depend on the surface area of the contact surface portion of the cap. A greater surface area of the contact surface may also help to further reduce the ability for the angle of incidence to vary from its desired value during use.

It will be appreciated that there are a number of body surfaces of a subject on which the cap and probe could be placed in order to take suitable measurements. For example, the body surface could be the skin, eye, tongue, nails, or any other internal or external body surface as appropriate. However, in some embodiments, the body surface comprises the subject's skin. For example, the cap and probe may be positioned on a patient's skin, such as on their hand, or on the skin of some other body part. The cap and probe may also be positioned on a wound, for example a non-healing wound such as a diabetic ulcer. Having a cap that is sterilised and that is disposable may be advantageous for such applications because, for example, the cap can be readily used with the probe for a given subject and disposed of before the probe is then used with a different subject.

The probe(s) may be used to determine a number of different haemodynamic parameters as is known in the art per se. However, the measured haemodynamic parameters may include at least one of: the concentration of haemoglobin in the subject's blood; the oxygenation level of the subject's blood; the local oxygenation level in the skin capillaries of the subject; the capillary density of the subject; and the capillary velocity of the subject. For example, the subject's microcirculation may be assessed in respect of the following parameters:
(a) functional capillary density (FCD);
(b) heterogeneity of the FCD;
(c) capillary flow velocity;
(d) heterogeneity of capillary flow velocity;
(e) oxygen saturation of microvascular erythrocytes (SmvO₂); and
(f) heterogeneity of SmvO₂;

Parameters (a) to (d) may be assessed by microscopy (e.g. wherein the microscope uses white, unpolarised light) while parameters (e) and (f) may be assessed by DRS. The assessment process is outlined in further detail in the Applicant's earlier publication WO 2014/114814, the contents of which are incorporated herein by reference.

While the contact surface and side wall are rigid to impede the angle of incidence from changing, the securing portion may in some arrangements be less rigid than the rest of the cap to allow for a removable connection between the cap and the probes. It will be understood that the term ‘removably secured’ as used herein means that the cap can be secured in place on the probe such that it does not readily come away from the probe during use (i.e. while measurements are being taken), but that the cap can be readily removed from the probe without breaking the cap or probe such that the cap can be re-used, e.g. with a different probe.

A further advantage associated with the cap of the present invention is that the contact surface may help to resist ‘slipping’ or ‘sliding’ of the probe relative to the skin, i.e. avoiding lateral movement of the probe in use. In other words, in addition to preventing changes in the measurement angle, the cap may also prevent changes in position of the probe.

The term ‘subject’ as used herein includes any human or non-human animal subject, including any human or non-human mammal, bird, fish, reptile, amphibian, etc. However, in preferred embodiments the subject is a human mammal, e.g. a human patient.

The present invention extends to a diagnostic method using the cap outlined hereinabove.

Thus, when viewed from a second aspect, the present invention provides a method for measuring haemodynamic parameters relating to a subject, the method comprising:
affixing a cap to a distal end of an optical probe;
positioning the optical probe such that the cap is positioned on a body surface of a subject; and
using the optical probe to take an optical measurement of at least one haemodynamic parameter relating to the subject;
wherein the cap comprises:
a rigid flat contact surface for contact with the body surface of the subject, said contact surface comprising an aperture, said aperture being arranged such that, in use, the aperture is optically aligned with the optical probe;
a rigid side wall surrounding the contact surface, wherein the side wall defines a closed end and an open end, said closed end being formed by the contact surface and said open end being arranged to receive the probe, said side wall being arranged such that, in use, the cap is held in abutment against the probe; and
a securing portion arranged to removably secure the cap to the probe.

This second aspect of the invention extends to a method for measuring haemodynamic parameters relating to a subject, the method comprising:
affixing a cap to a distal end of a DRS probe;
positioning the DRS probe such that the cap is positioned on a body surface of a subject;
using the DRS probe to take at least one DRS measurement of at least one haemodynamic parameter relating to the subject;
removing the cap from the distal end of the DRS probe;
affixing the cap to a distal end of a microscope probe;
positioning the microscope probe such that the cap is positioned on the body surface of the subject;
using the microscope probe to take at least one microscope measurement of at least one haemodynamic parameter relating to the subject;
wherein the cap comprises:
a rigid flat contact surface for contact with a body surface of the subject, said contact surface comprising an aperture, said aperture being arranged such that, in use, the aperture is optically aligned with the optical probe in use;
a rigid side wall surrounding the contact surface, wherein the side wall defines a closed end and an open end, said closed end being formed by the contact surface and said open end being arranged to receive the probe in use, said side wall being arranged such that, in use, the cap is held in abutment against the probe; and
a securing portion arranged to removably secure the cap to each of the probes.

In some embodiments, the method is a non-invasive method. In other embodiments, the method is an invasive method.

In some embodiments, the method further comprises applying an oil, for example baby oil, to the body surface of the subject prior to positioning the microscope probe on the body surface. As outlined above, the use of oil such as baby oil may help to reduce specular reflections during the microscopy procedure. The oil may be clear (i.e. substantially optically transparent) and skin-compatible, and may comprise paraffinum liquidum (i.e. liquid paraffin) and/or isopropyl palmitate, though it is envisaged that other suitable oils may be used as appropriate.

In some embodiments, the method further comprises using the at least one DRS measurement and the at least one microscope measurement to determine one or more haemodynamic parameters relating to the subject. In some such embodiments, the method further comprises determining an indication relating to the subject from the one or more haemodynamic parameters. This determination may, in some examples, be made by comparing the measurements from the probe(s) to reference values.

The method may, in some embodiments, be a method of identifying or monitoring circulatory failure in a subject.

One or more steps of the method may, in some embodiments, be partially or wholly computer-implemented.

In some embodiments in which a microscope probe is used, the microscope may be a computer assisted video microscope (CAVM).

The area of the contact surface may be selected as appropriate, however in some embodiments an area of the contact surface is at least approximately 4 cm², preferably at least approximately 5 cm², and more preferably is at least approximately 6 cm². For example, the area of the contact surface may be between approximately 4 cm² and 10 cm², optionally between approximately 6 cm² and 8 cm², and may, for example, be approximately 6.6 cm². A greater surface area may reduce the pressure on the body surface and may improve the stability of the angle of incidence when compared to the conventional approach where the contact area with the body surface may be approximately 1-2 cm² as outlined above; however, this comes at a trade-off for a larger cap which would therefore require additional material, adding to the cost of the cap. The contact surface may, as outlined below, be of substantially circular cross-section, and may have a diameter between approximately 20 mm and 40 mm, for example a diameter of approximately 30 mm.

The optimal angle of incidence for a given probe will generally depend on the design of the probe itself, however where the probe is to be held normal (i.e. at right angles to) the body surface, the cap may retain the probe in an upright position. Thus, in use, the probe may extend in a direction normal to the contact surface of the cap.

The aperture allows light to pass through the cap to and/from the probe as appropriate, thereby allowing measurements such as DRS and microscopy measurements to be taken. While the aperture could be left as an open ‘hole’ that passes through the contact surface, in some embodiments the cap further comprises a substantially transparent window portion that covers the aperture of the contact surface. The window portion seals the aperture, thereby advantageously preventing the ingress of dirt, grease, particulate matter etc. into the interior of the cap. This may, for example, prevent contaminates affecting the optics of the probe(s) when positioned within the cap. This may be particularly advantageous where the subject's body surface is coated with baby oil, for example during a microscopy procedure. In some embodiments, the window portion is rigid.

Sealing the aperture with a window portion may also advantageously prevent the body part under examination from contamination, e.g. due to pathogens, cleaning agent residues, chemicals, or dirt on the optical probe(s).

It will be appreciated that the term ‘substantially transparent’ means that the window portion is sufficiently optically transparent to electromagnetic radiation having a wavelength used by the probe(s). For example, where the cap is used with a DRS probe and/or an microscope, the window portion is sufficiently transparent that visible and NIR light may readily pass through the window portion, thereby permitting measurements to be taken with the probe. The Applicant has appreciated that, where an emitting probe is used (i.e. a probe that emits light such as a DRS probe), the distance between the operating end of the probe and the window portion may affect the degree to which the window portion gives rise to specular reflections of the light emitted by the probe. As such, in some embodiments the cap is arranged such that, in use, a distance between the probe and the window portion is less than approximately 2 mm, is preferably less than approximately 1.5 mm, and is more preferably less than approximately 1 mm, for example between approximately 0.5 mm and 1 mm. In preferred embodiments, the cap is arranged such that, in use, a distance between the probe and the window portion is less than approximately 0.5 mm. Such a small distance between the optics of the probe (i.e. the ‘operating end’) and the window portion may reduce the effect of specular reflections (akin to shining a flashlight next to a glass window) arising from the incident light produced by the probe. In some embodiments, the cap is arranged such that, in use, the operating end of the probe is in contact with the transparent window portion.

The window portion may, in some embodiments, have a thickness less than 1 mm, for example between approximately 0.10 mm and 0.80 mm, for example between approximately 0.20 mm and 0.60 mm, optionally between approximately 0.25 mm and 0.50 mm. In some embodiments, the thickness of the window portion may be approximately 0.25 mm or 0.50 mm.

The aperture of the contact surface may be sized as appropriate for the probes it is to be used with. However, in some embodiments the aperture has a diameter between approximately 5 mm and 15 mm, optionally between approximately 7.5 mm and 12.5 mm, and may for example be approximately 10 mm.

In some embodiments, the contact surface and side wall are optically opaque, i.e. they substantially prevent ambient light from passing through the cap to the probe, e.g. into the optics or an optical fibre of the probe. For example, in some such embodiments, the contact surface and side wall are black. However, it will be appreciated that these may be optically opaque without being black, e.g. with use of sufficiently optically absorbent materials. Similarly, the securement portion may, at least in some potentially overlapping embodiments, be optically opaque, e.g. black.

The cap may have any suitable shape, where its shape will depend on the shape of the probes with which it is intended to be used. However, in at least some embodiments, the cap is substantially cylindrical. In other words, the contact surface may be circular, with the side wall having a circular cross-section extending from the contact surface.

The side wall may have the same diameter as the contact surface, however in some embodiments the side wall has a diameter less than a diameter of the securement portion. In other words, the side wall may be narrowed compared to the securement portion. This may provide a ‘shoulder’ on which the probe rests, i.e. the probe is brought into abutment with the side wall at the shoulder, where the optics of the probe may extend into the narrowed side wall cavity, toward the aperture. Thus the probe may enter the cap at the open end, where the securement portion is sufficiently wide to allow the probe to enter the cap, wherein the shoulder provided by the narrowed side wall prevents the probe passing any further into the cap. Depending on the choice of dimensions of the cap (e.g. the height of the cap, the height of the side wall, and the design of the probe such as how far its optics protrude from the probe), the shoulder may set the gap between the optics of the probe and the aperture, as outlined in further detail below.

Similarly, the diameter of the side wall may, in some potentially overlapping embodiments, be less than a diameter of the contact surface. Where the side wall is narrower than both the contact surface and securement portion, the side wall is effectively a ‘collared’ part of the cap, i.e. the cap narrows in the region of the side wall. Thus the side wall may be sufficiently narrow to set the distance between the probe and the aperture of the contact surface, but the contact surface is not limited to the diameter of the of the side wall.

There are a number of materials, known in the art per se, that could be used to form the cap. However, in some embodiments, the contact surface and the side wall comprise a polymer. In a preferred set of such embodiments, the contact surface and the side wall comprise the same polymer. In a set of potentially overlapping embodiments, the securing portion comprises a polymer, and preferably comprises the same polymer as the contact surface and/or side wall. Preferably, the polymer is suitable for an injection molding process. Those skilled in the art will appreciate that there are a number of polymers suitable for injection molding that could provide the required rigidity. The polymers may, for example, be thermosetting or thermoplastic polymers.

Other suitable manufacturing processes may include extrusion molding and/or blow molding.

Preferably, the polymer is biocompatible, i.e. it is not harmful or toxic to the subject. Additionally, it is preferable that the polymer does not discolour when sterilised, which is important as it is envisioned that the cap will typically be sterilised prior to use, e.g. during the manufacturing process. Where different polymers are used for the cap body and window portion, it is advantageous for both polymers to have one or more of these properties.

By way of non-limiting example, such suitable polymers include for example, polyesters, copolyesters, and polycarbonates.

Polycarbonates may be linear or branched, and may comprise units derived from one or more bisphenols, e.g. bisphenol A. Examples of such materials include the polycarbonate resins available from Bayer, USA under the tradename Makrolon. These are bisphenol-based homopolycarbonate resins having varying molecular weights. An example of such a material is Makrolon 2458.

Polyesters which may be used include the copolyester resins available from Eastman Chemical Company, USA, for example Eastar MN200.

It is also preferable that the polymer does not discolour when stored.

In a set of potentially overlapping embodiments, the contact surface and the side wall are integrally formed. Similarly, in a set of potentially overlapping embodiments, the securing portion and the side wall are integrally formed. While the parts of the cap could be formed independently and joined together later, by having the parts of the cap integrally formed, the body of the cap (i.e. the contact surface, side wall, and securing portion) can be made in a single manufacturing step. For example, injection molding can be used to form the body of the cap. The ability to manufacture the cap by injection molding is particularly desirable as this manufacturing method can be used to produce high volumes of the caps (i.e. it provides for mass production). As the caps are a medical device that are typically used once and disposed of, it is advantageous to produce high volumes of the caps at low cost.

In embodiments comprising the transparent window portion, the window portion could be made from a different material than the rest of the cap, in some embodiments the window portion comprises the same material as the contact surface and side wall, and optionally the securing portion. The Applicant has appreciated that it is particularly advantageous from a manufacturing process to have the window portion be constructed from the same material as the body of the cap. This is because having these made from the same material may help to ensure that both the window portion and the surrounding cap body have the same shrinking properties after cooling down. If different materials are used, this may lead to fractures and cracks in the materials. However, this is not essential as other steps may be taken to avoid such issues.

In some embodiments, the polymer is initially transparent and colouring is added during manufacturing to produce the contact surface and side wall portions of the cap. This may be advantageous where the same polymer is used for both the cap body and the window portion, as colouring can be added to the polymer used for the body of the cap while the polymer used for the window portion can be left transparent.

The window portion could be integrally formed with the contact surface, however in some embodiments the window portion is affixed to the surface portion, e.g. using glue or ultrasonic welding. Such embodiments may simplify the manufacturing process, for example by allowing the use of a single transparent polymer for all parts of the cap, where colouring is added to the polymer for the main cap body while the polymer used for the window portion is kept transparent, without needing to prevent the colouring affecting the window portion.

As outlined above, the securing portion acts to retain the cap against the probe when in use. This may be achieved with an interference fit, by using magnets, or through the use of a fastening mechanism such as a threaded connector (akin to a nut-and-bolt). However, in a set of embodiments, the securing portion comprises a plurality of protrusions extending from the side wall, wherein each of said protrusions comprises an engagement member arranged to engage with a corresponding engagement member on the probe.

This is novel and inventive in its own right and thus, when viewed from a third aspect, the present invention provides a cap for use at a distal end of an optical probe, wherein the cap comprises:
a flat contact surface for contact with a body surface of a subject, said contact surface comprising an aperture, said aperture being arranged such that, in use, the aperture is optically aligned with the optical probe;
a side wall surrounding the contact surface, wherein the side wall defines a closed end and an open end, said closed end being enclosed formed by the contact surface and said open end being arranged to receive the probe, said side wall being arranged such that, in use, the cap is held in abutment against the probe; and
a securing portion arranged to removably secure the cap to the probe, the securing portion comprising a plurality of protrusions extending from the side wall, wherein each of said protrusions comprises an engagement member arranged to engage with a corresponding engagement member on the probe.

This third aspect of the invention extends to an optical probe arrangement for measuring haemodynamic parameters relating to a subject, the system comprising:
an optical probe; and
a cap for use at said distal end of the probe, wherein the cap comprises:
a flat contact surface for contact with a body surface of a subject, said contact surface comprising an aperture, said aperture being arranged such that, in use, the aperture is optically aligned with the optical probe;
a side wall surrounding the contact surface, wherein the side wall defines a closed end and an open end, said closed end being enclosed formed by the contact surface and said open end being arranged to receive the probe, said side wall being arranged such that, in use, the cap is held in abutment against the probe; and
a securing portion arranged to removably secure the cap to the probe, the securing portion comprising a plurality of protrusions extending from the side wall, wherein each of said protrusions comprises an engagement member arranged to engage with a corresponding engagement member on the probe.

This third aspect of the invention further extends to a system for measuring haemodynamic parameters relating to a subject, the system comprising:
a DRS probe;
a microscope probe; and
a cap for use at a respective distal end of each of the probes, one at a time, wherein the cap comprises:
a flat contact surface for contact with a body surface of a subject, said contact surface comprising an aperture, said aperture being arranged such that, in use, the aperture is optically aligned with the optical probe in use;
a side wall surrounding the contact surface, wherein the side wall defines a closed end and an open end, said closed end being enclosed formed by the contact surface and said open end being arranged to receive the probe, said side wall being arranged such that, in use, the cap is held in abutment against the probe; and
a securing portion arranged to removably secure the cap to the probe, the securing portion comprising a plurality of protrusions extending from the side wall, wherein each of said protrusions comprises an engagement member arranged to engage with a corresponding engagement member on each of the probes.

In some embodiments, the engagement member of the cap may be a tab, a rib, a tongue, or any other suitable engagement member that can be brought into engagement with a corresponding engagement on the probe, which may be a groove, a socket, a hole, or any other suitable corresponding engagement member. The corresponding engagement member (e.g. a groove) on the probe may, for example, be located on a handle of the probe, and may extend around part or all of an exterior surface of the probe, e.g. around the circumference of the probe handle. In such arrangements, the engagement member of the cap can be seen as the ‘male’ and the engagement member of the probe as the ‘female’. However, the ‘male’ and ‘female’ roles of the engagement members could be reversed, e.g. where at least one groove or socket is provided on the securing portion of the cap, and the corresponding engagement members of the probe comprise suitable tab(s), rib(s), tongue(s), etc.

Thus with proper dimensioning of the cap, e.g. the height of the side wall and the relative positioning of the engagement members and the corresponding engagement members on the probe, such embodiments may provide a convenient ‘clip-on’ feature, whereby the cap may be readily attached and detached from probes (e.g. a DRS probe and a microscope probe) between uses. Such an arrangement may also advantageously help to provide a secure connection between the cap and the probe, thus helping to prevent the cap from shifting position in use and thereby aiding in the prevention of the probe being moved from its proper alignment (i.e. preventing any change in the angle of incidence), while allowing for disconnection without breaking the cap or probe.

In some embodiments, the securement portion is less rigid than the contact surface and side wall. For example, the protrusions may be less rigid than the side wall and contact surface. The reduced rigidity of the securement portion may help to facilitate the ‘click on’ function of the cap.

The method of manufacturing the cap is also considered novel and inventive in its own right. Thus, when viewed from a fourth aspect, the present invention provides a method of manufacturing a cap for use at a distal end of an optical probe, the method comprising:
injection molding a first polymer to produce a cap body comprising:
   a flat contact surface for contact with a body surface of a subject, said contact surface comprising an aperture, said aperture being arranged such that, in use, the aperture is optically aligned with the optical probe in use;
   a side wall surrounding the contact surface, wherein the side wall defines a closed end and an open end, said closed end being formed by the contact surface and said open end being arranged to receive the probe, said side wall being arranged such that, in use, the cap is held in abutment against the probe; and
   a securing portion arranged to removably secure the cap to the probe;
the method further comprising:
injection molding a second polymer to produce a substantially transparent window portion; and
affixing the window portion to the contact surface such that the window portion covers the aperture of the contact surface.

As outlined above, the first and second polymers may be different polymers or may be the same polymer. Where these are the same polymer, the method may, at least in some embodiments, further comprise:
adding a colourant to a transparent polymer thereby producing the first polymer; and
using the transparent polymer as the second polymer. It will be appreciated that, in accordance with such embodiments, the transparent polymer may be used directly as the second polymer, but it is envisaged that further processing steps may be carried out on it to make it more suitable for passage of electromagnetic radiation (i.e. light). The result is that the first polymer has a greater optical opacity than the second polymer.

The optional features described hereinabove in relation to any particular aspect of the invention also apply to the other aspects of the invention in any combination as appropriate.

### BRIEF DESCRIPTION OF THE DRAWINGS

Certain embodiments of the present invention will now be described with reference to the accompanying drawings, in which:

FIG. 1 is a side-on view of the body of a cap in accordance with an embodiment of the present invention;

FIG. 2 is a perspective view of the body of the cap of FIG. 1, providing a view of the interior of the cap;

FIG. 3 is a further view of the body of the cap of FIG. 1, providing a view of the side profile of the cap;

FIG. 4 is a schematic drawing of a window portion to be used with the cap body of FIGS. 1 to 3;

FIG. 5 is a cutaway view of a cap including both the cap body and window portion once assembled;

FIG. 6 shows a system for taking measurements of haemodynamic parameters relating to a patient;

FIG. 7 shows the system of FIG. 6 when the DRS probe is in use;

FIG. 8 shows the system of FIG. 6 when the microscope probe is in use;

FIG. 9 is a chart that shows the O₂ estimate for varying degree of probe tilt;

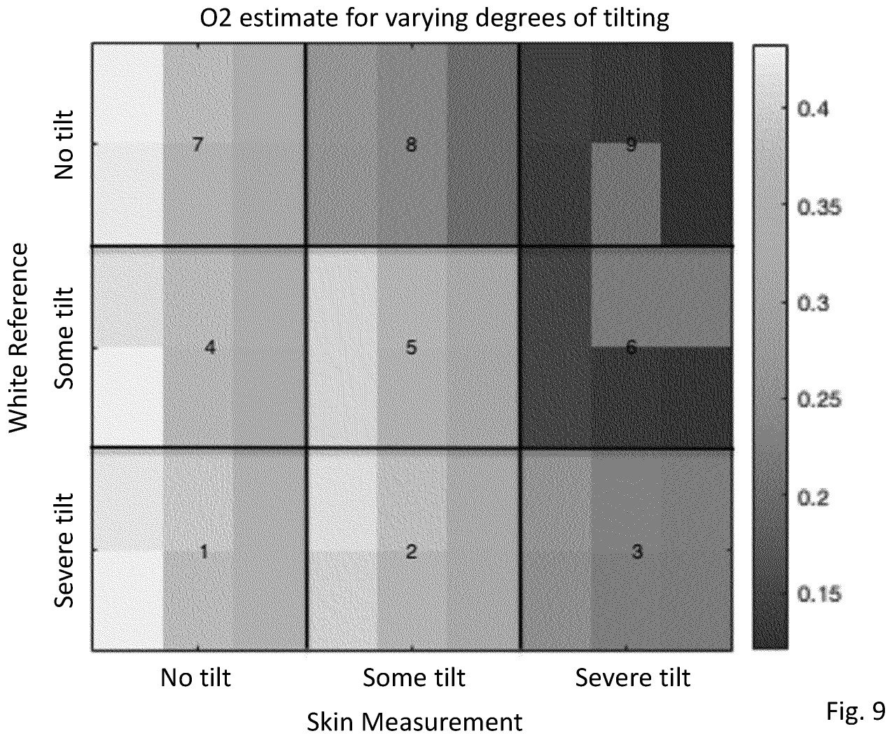

FIG. 10 is a box plot that shows the mean intensity of reflected light associated with a number of different cap arrangements; and

FIG. 11 is a box plot that shows an exemplary set of O₂ saturation measurements acquired with a DRS probe using different cap arrangements.

### DETAILED DESCRIPTION

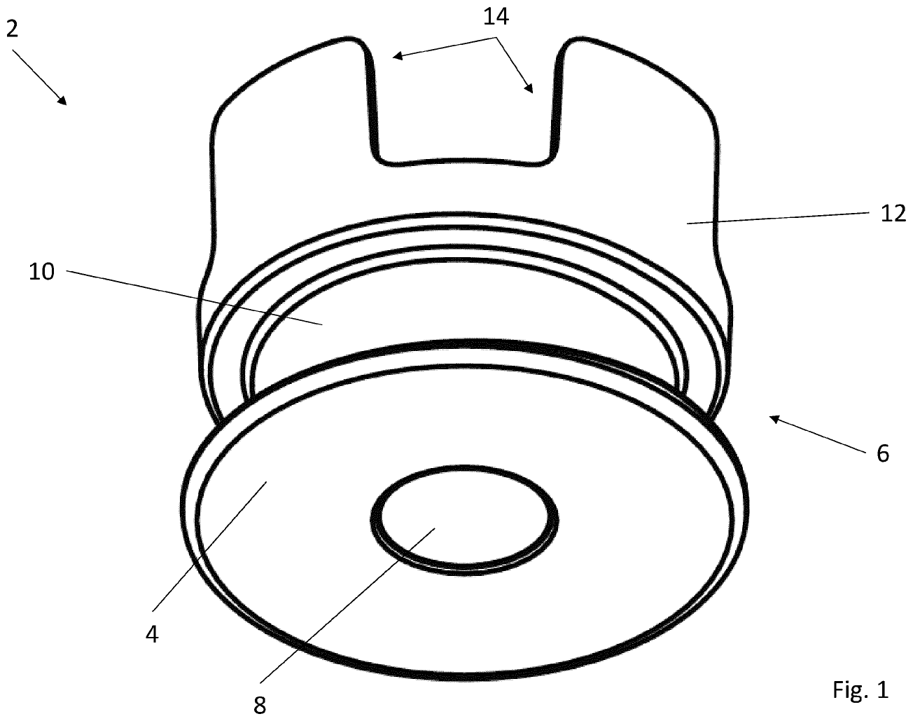

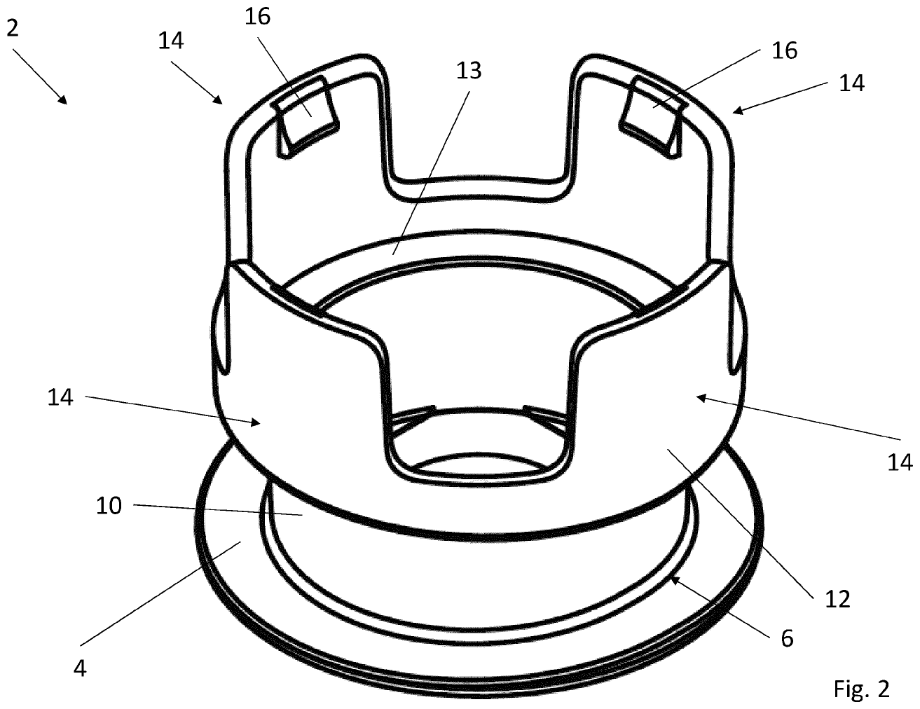

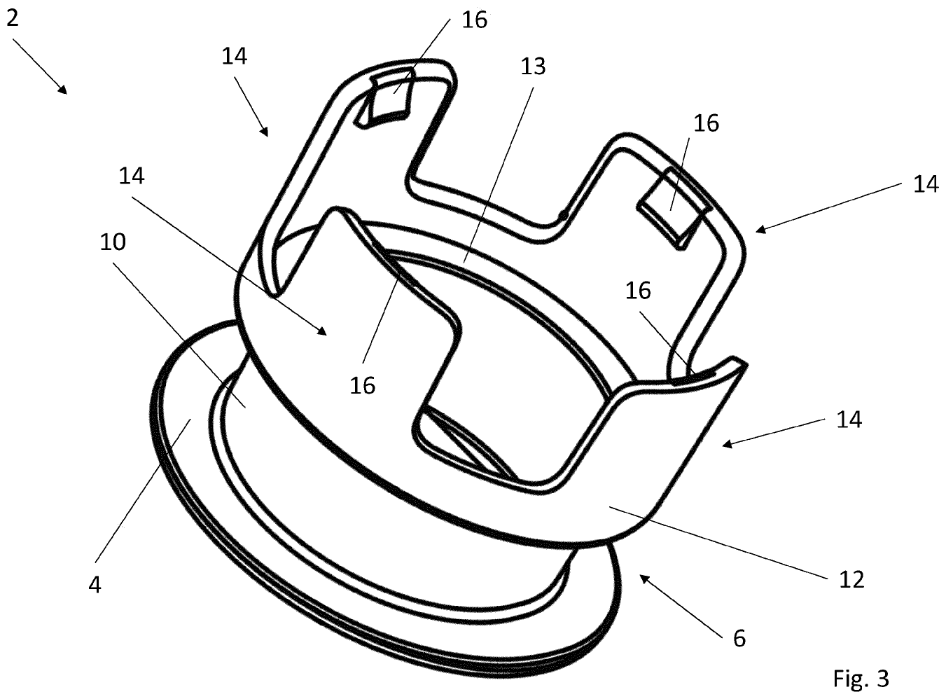

FIGS. 1 to 3 show the body of a cap 2 in accordance with an embodiment of the present invention in different views. Specifically, FIG. 1 is a side-on view of the body of the cap 2; FIG. 2 is a perspective view of the body of the cap 2, providing a view of the interior of the cap 2; and FIG. 3 is a further view of the body of the cap 2, providing a view of the side profile of the cap 2.

The cap 2 comprises a contact surface 4 and a side wall 6. As can be seen clearly in FIG. 1, the contact surface 4 is substantially flat and has a circular cross-section. The centre of the contact surface 4 is provided with a circular aperture 8. The contact surface 4 and side wall 6 are both constructed from an optically opaque polymer, and are formed using injection molding. The polymer used may, for example, be Eastar MN200, polycarbonate, or Makrolon 2458. These polymers may initially be transparent, where a colourant is added to provide the desired optical opacity prior to the injection molding process.

The side wall 6 is of a substantially cylindrical construction, such that the side wall extends around the contact surface 4. A substantially cylindrical securement portion 12 extends from the top of the side wall 6, where the securement portion 12 is discussed in further detail below. The side wall 6 has an annular portion 10 which is of smaller diameter than the securement portion 12 and the contact surface 4, though it will be appreciated that other arrangements are possible. The narrowed annular portion 10 provides a shoulder 13 on which the probe can rest.

At the securement portion 12 located at the top of the side wall 6, i.e. at the open end of the cap 2, there are a number of protrusions 14 that extend away from the contact surface 4. In this particular example, the protrusions 14 have a ‘bunny ear’ shape, though it will be appreciated that alternative shapes could be used as appropriate. The cap 2 of FIGS. 1 to 3 has four such protrusions 14.

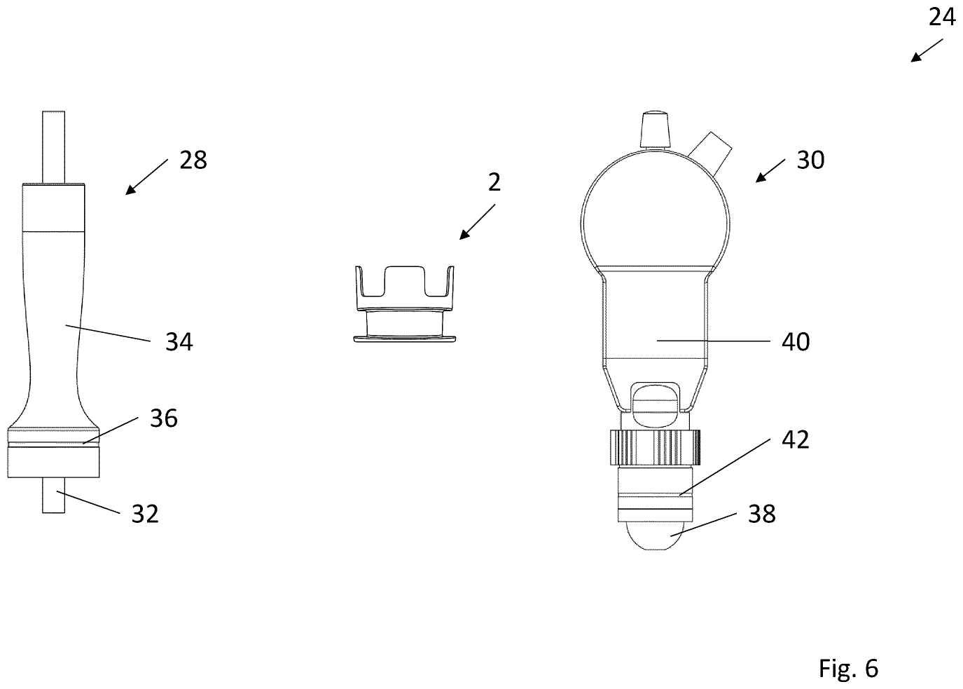

Each of the protrusions 14 has an engagement member 16 which is arranged to engage with corresponding engagement member (e.g. a groove) on the probe when in use, as described in further detail below with reference to FIGS. 6 to 8. In this example, the engagement members 16 are small tabs that extend from the respective protrusion 14 toward the centre of the cap 2, i.e. in the direction of the aperture 8. For the sake of simplicity, all of the engagement members 16 are of identical dimensions and are positioned at the same height, i.e. distance from the contact surface 4, thus allowing easy connection and disconnection from a probe with a uniform groove around its handle. However, other arrangements are envisaged where this is not necessary, e.g. tabs at different heights arranged to engage with groove(s) at different positions on the probe handle.

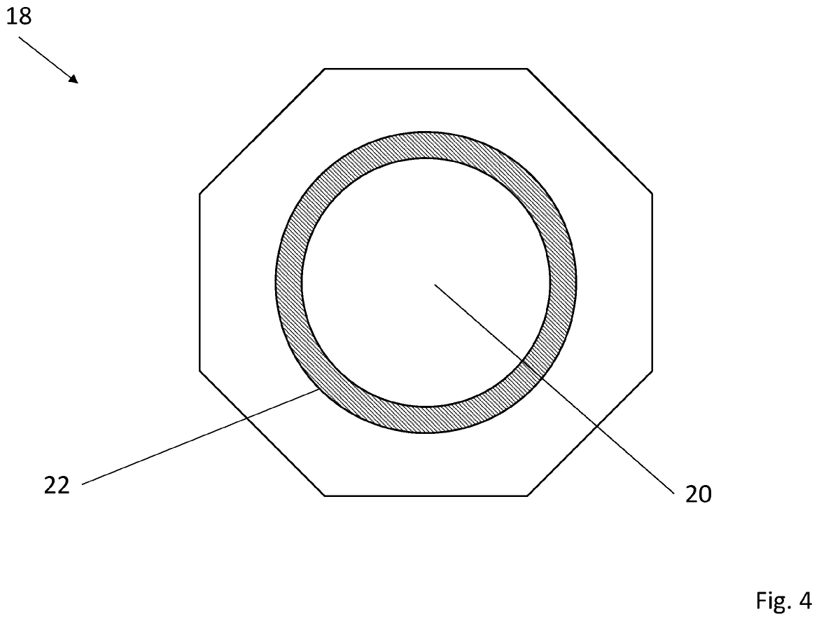

FIG. 4 is a schematic drawing of a window portion 18 to be used with the cap body of FIGS. 1 to 3. The window portion 18 is constructed from a transparent polymer, such that it is substantially transparent with respect to at least visible and NIR light, e.g. to wavelengths between approximately 400 nm and 1400 nm. The window portion 18 may, of course, have different optical properties, depending on the requirements of the probes with which the cap 2 is to be used.

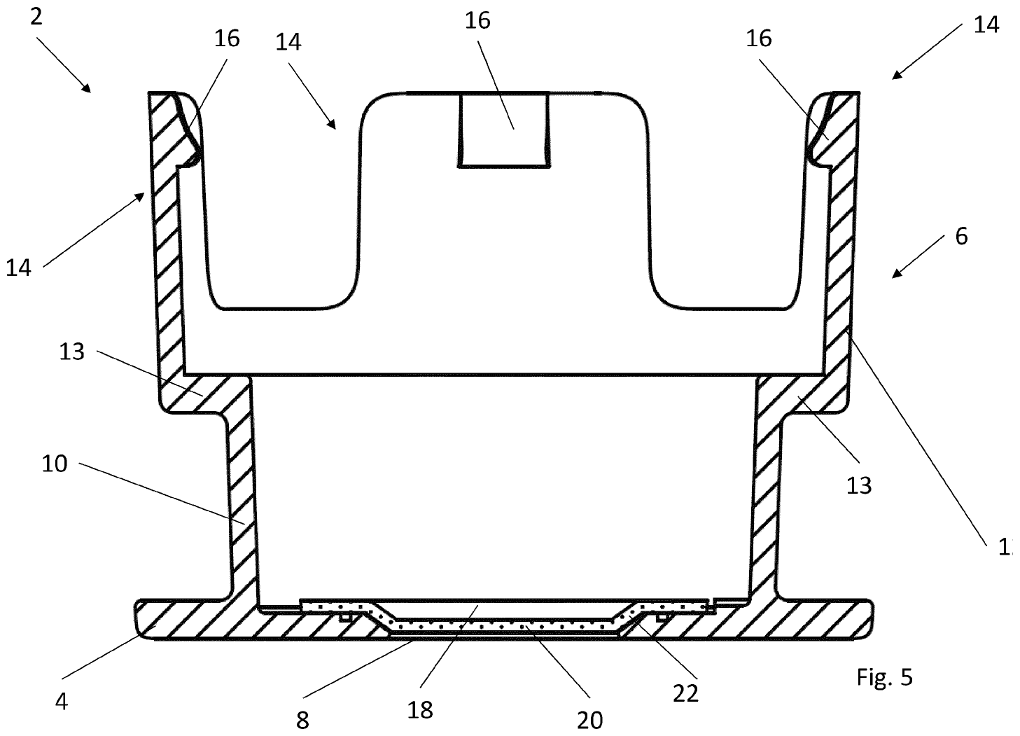

The window portion 18 has a central part 20 and a peripheral part 22. The central part 20 is the primary area through which light travels, e.g. during DRS or microscopy measurements. The peripheral part 22 surrounds the central part 20 and is shaped to fit in a suitable recess provided in the contact surface 4, as can be seen more clearly in FIG. 5.

FIG. 5 is a cutaway view of a cap 2 including both the cap body (i.e. the contact surface 4 and side wall 6) and window portion 18 once assembled. As can be seen in FIG. 5, the window portion 18 is positioned such that the aperture 8 is covered by the window portion 18. Thus the window portion 18 seals the aperture 8, thereby preventing the ingress of dirt, grease, particulate matter etc. into the interior of the cap 2. This prevents contaminates affecting the optics of a probe positioned within the cap 2, which may be particularly useful for microscopy procedures where baby oil is used, because the cap 2 prevents the oil getting to the microscope optics while allowing light to pass through the contact surface 4 via the window portion 18.

Similarly, sealing the aperture 8 with a window portion 18 may also advantageously prevent the body part under examination from contamination, e.g. due to pathogens, cleaning agent residues, chemicals, or dirt on the probe(s).

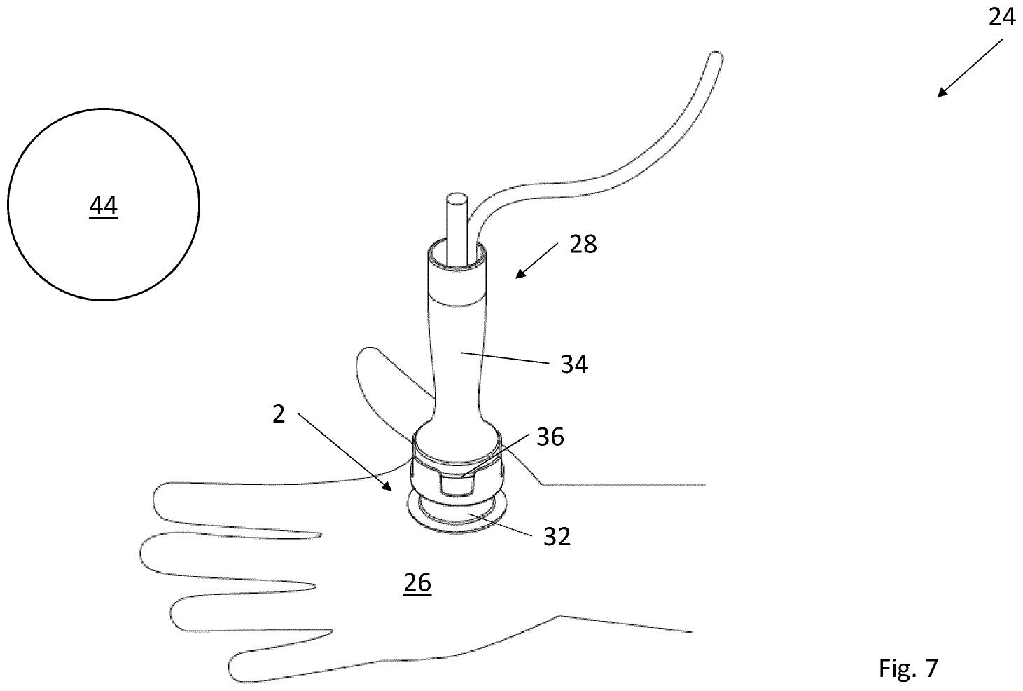

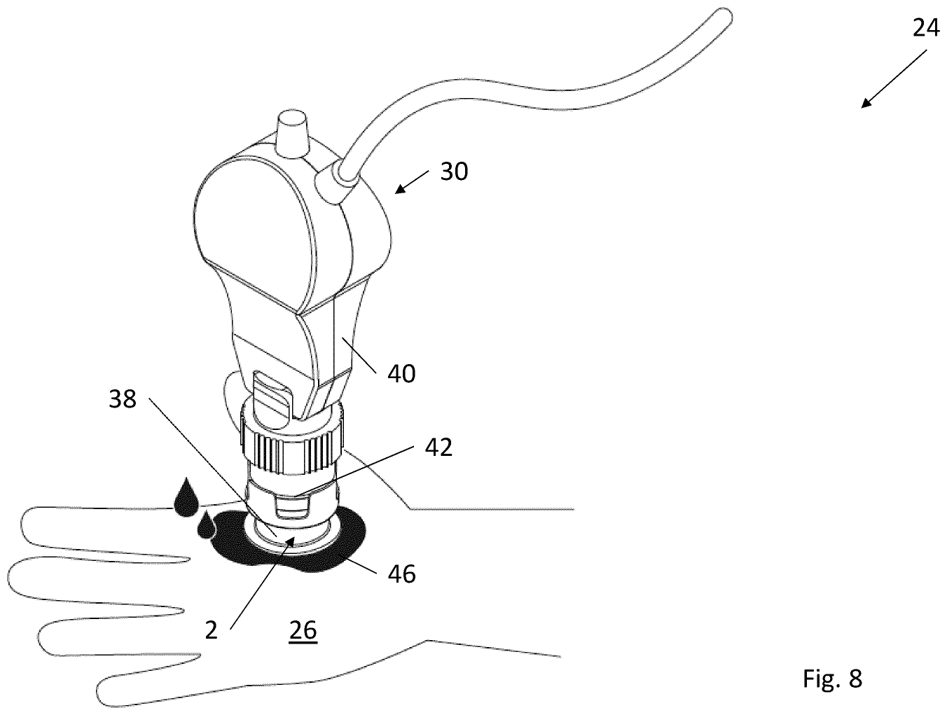

FIG. 6 shows a system 24 for taking measurements of haemodynamic parameters relating to a patient 26. The system 24 comprises a DRS probe 28 and a video microscope probe 30. The system 24 also includes the cap 2 described hereinabove with reference to FIGS. 1 to 5. In FIG. 6, the system 24 is shown without the cap 2 engaged with either probe 28, 30, however the cap 2 is shown in use in FIGS. 7 and 8, which are described in further detail below.

The DRS probe 28 includes optics 32, which are contained within a handle 34, which acts as a housing for the probe 28. The handle 34 has a groove 36 that extends around the periphery of the handle 34.

Similarly, the microscope probe 30 includes optics 38, which are contained within a handle 40, which acts as a housing for the probe 30. The handle 40 also has a groove 42 that extends around the periphery of the handle 40.

Both probes 28, 30 are to be used on the skin on the patient's hand 26 to determine haemodynamic parameters relating to the patient, where the DRS and microscopy measurement steps are described with reference to FIGS. 7 and 8 below respectively. It will be appreciated that while the hand of the patient 26 is used in this example, other parts of the body could be used instead as appropriate.

FIG. 7 shows the system 24 of FIG. 6 when the DRS probe 28 is in use. The cap 2 is ‘clicked’ into place on the DRS probe 28, where the engagement members 16 on the protrusions 14 physically engage with the groove 36 on the handle 34 of the DRS probe 28. Once put in place, the gap between the optics 32 of the DRS probe 28 and the transparent window portion 18 of the cap 2 is minimal, for example less than 0.5 mm, and the optics 32 of the DRS probe 28 may be pushed right up against the window portion 18 in some arrangements.

Firstly, a measurement is taken in which the DRS probe 28 is placed against a white reference 44, which substantially reflects all light. The measurements taken in relation to the white reference 44 are used for the sake of comparison. Generally, the operator may take a number of measurements using the white reference 44, for example three measurements may be taken using the white reference 44.

The operator then moves the DRS probe 28 to the patient's skin 26 and takes a number of DRS measurements relating to the patient. For example, the operator may take twelve DRS measurements in respect of the patient's skin 26.

The DRS recordings are captured from a skin area of roughly 1-2 cm². The DRS probe 28 is relocated for each of the twelve recordings in order to provide statistical data for averaging and calculation of coefficient of variation. From these recordings, the level of local oxygenation in the skin capillaries is extracted by a suitable algorithm, known in the art per se, the details of which are not described herein.

The DRS probe 28 is kept substantially upright throughout the DRS measurements because the flat contact surface 4 of the cap 2 which is in contact with the patient's skin 26 impedes the tilting of the probe 28 away from the optimum angle of incidence, which in this example is normal (i.e. at right angles) to the patient's skin 26. This helps to improve the reliability of repeatability of the measurements taken with the DRS probe 28.

Once all of the DRS measurements have been taken, the video microscopy steps are carried out as described with reference to FIG. 8, which shows the system 24 of FIG. 6 when the microscope probe 30 is in use.

The cap 2 is removed from the DRS probe 28. While the mechanical connection between the cap 2 and the probes 28, 30 is robust during use, the ‘click-on’ nature of the cap 2 makes it relatively easy for the operator to remove it from one probe and to move it over to the other probe.

Once removed from the DRS probe 28, the cap 2 is ‘clicked’ into place on the microscope probe 30. Similarly to with the DRS probe 28, the engagement members 16 on the protrusions 14 physically engage with the groove 42 on the handle 40 of the microscope probe 30. The gap between the optics 38 of the microscope probe 30 and the transparent window portion 18 of the cap 2 is minimal, for example less than 0.5 mm, and the optics 38 of the microscope probe 30 may be pushed right up against the window portion 18 in some arrangements.

Baby oil 46 is applied to the patient's skin 26 in order to reduce the impact of specular reflections on measurements taken with the microscope probe 30. The video microscope 30 is then positioned on the patient's skin 26 and a number of video recordings are taken. Generally, the use of the baby oil 46 in the microscopy procedure means that the microscopy is typically carried out after the DRS procedure, which requires no such oil.

In this particular example, five videos are recorded using the microscope probe 30 which captures a video sequence of 20 seconds for each of the recordings. As with the DRS procedure described above with reference to FIG. 7, the microscope probe 30 is relocated for each recording and the total area covered is approximately the same as in the DRS procedure. From these video recordings, the capillary density (i.e. the capillaries per square millimeter which may be counted by human visual analysis) and velocities are extracted (which may be graded on a scale ranging from 0-5 by a human analysis and interpretation).

The cap 2 prevents the baby oil 46 from coming into contact with the optics 38 of the microscope probe 30. This may advantageously reduce the amount of cleaning of the probe 30 that is required, and may help to prevent the ingress of grease, dirt, etc. from affecting the operation of the microscope probe 30. As above, sealing the aperture 8 with a window portion 18 may also advantageously prevent the body part under examination from contamination, e.g. due to pathogens, cleaning agent residues, chemicals, or dirt on the microscope probe 30.

As with the DRS probe 28, the microscope probe 30 is kept substantially upright during the measurements due to the flat contact surface 4 of the cap 2. This helps to improve the reliability of repeatability of the measurements taken with the microscope probe 30.

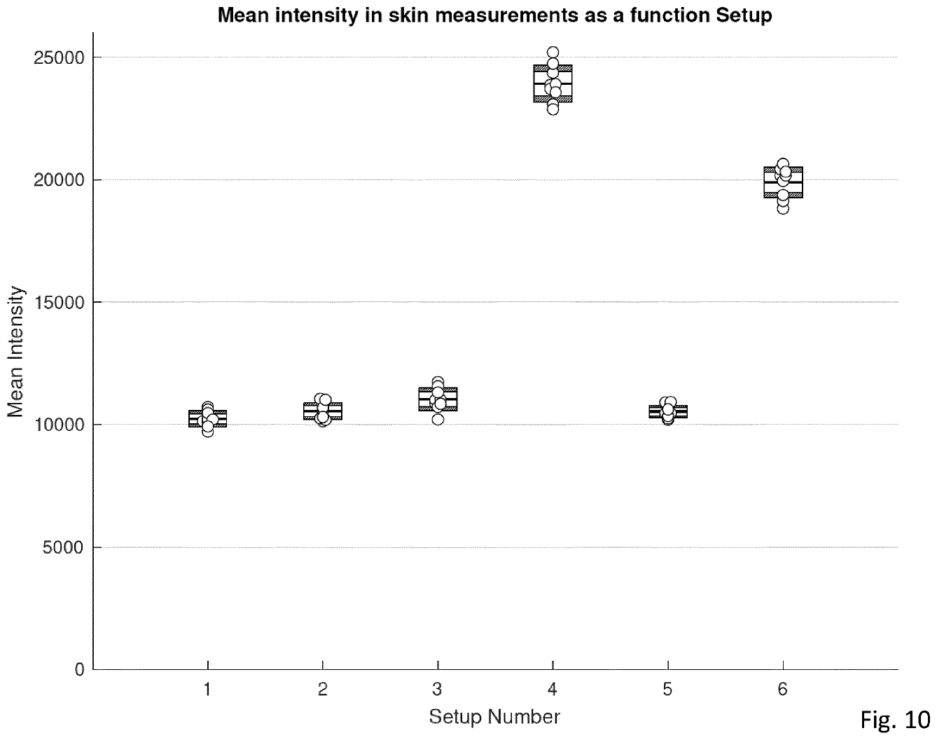

FIG. 10 is a box plot that shows the mean intensity of reflected light associated with a number of different cap arrangements. Specifically, FIG. 10 shows the mean intensity over a recorded spectrum of 400 nm to 900 nm as a function of different cap setups.

There are nine recordings for each of the six setups, where each recording is depicted as a dot on the corresponding box plot. The central line across the box plot for each setup indicates the mean value associated with that cap setup. The area of the box immediately outwards of the central line shows the standard error of the mean (95% confidence interval) for that setup, assuming a Gaussian distribution of the measurements. The outer box for each setup indicates the standard deviation.

In particular, the arrangements used in this pilot study, shown in FIG. 10 in the following order are:
1. A bare probe without a cap.
2. A flexible black cap.
3. A rigid black cap with window of 0.25 mm thickness, where the probe is pushed probe onto the window portion.
4. A rigid black cap with window of 0.25 mm thickness, where there is a gap of 0.5 mm to 1.0 mm between the probe and the window portion.
5. A rigid black cap with window of 0.50 mm thickness, where the probe is pushed probe onto the window portion.
6. A rigid black cap with window of 0.50 mm thickness, where there is a gap of 0.5 mm to 1.0 mm between the probe and the window portion.

FIG. 10 shows the mean intensity from the spectroscopy recording on skin using the various cap setups as described above. It can be seen from the box plot that the average intensity only changes significantly in setups where there is a window with a distance of 0.5 mm to 1.0 mm between the probe tip and the window (i.e. measurements 4 and 6).

Thus measurements 4 and 6 demonstrate the ‘flash light’ effect referred to previously. However, where the probe tip is pushed close to the transparent window portion (i.e. measurements 3 and 5), it is clear that the window portion does not significantly impact to average intensity of the reflected light compared to cases where no window is used (i.e. measurements 1 and 2).

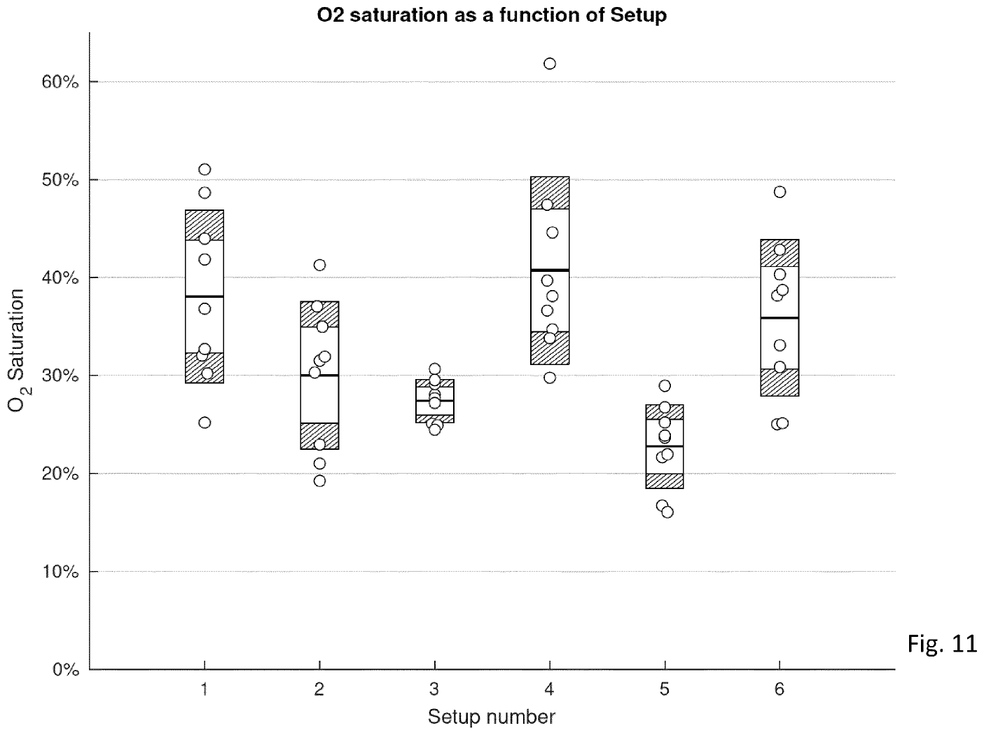

FIG. 11 is a box plot that shows an exemplary set of O₂ saturation measurements acquired with a DRS probe using different cap arrangements.

There are nine recordings for each of the six setups, where each recording is depicted as a dot on the corresponding box plot. The central line across the box plot for each setup indicates the mean value associated with that cap setup. The area of the box immediately outwards of the central line shows the standard error of the mean (95% confidence interval) for that setup, assuming a Gaussian distribution of the measurements. The outer box for each setup indicates the standard deviation.

In particular, the arrangements used in this pilot study, shown in FIG. 11 in the following order are:
1. A bare probe without a cap.
2. A flexible black cap.
3. A rigid black cap with window of 0.25 mm thickness, where the probe is pushed probe onto the window portion.
4. A rigid black cap with window of 0.25 mm thickness, where there is a gap of 0.5 mm to 1.0 mm between the probe and the window portion.
5. A rigid black cap with window of 0.50 mm thickness, where the probe is pushed probe onto the window portion.
6. A rigid black cap with window of 0.50 mm thickness, where there is a gap of 0.5 mm to 1.0 mm between the probe and the window portion.

When no cap or a flexible cap was used (measurements 1 and 2 respectively), the results show variable oxygenation and a relatively large variance.

When a stiff cap was used with a window but there is an extra gap (of 0.5 mm to 1.0 mm) between the tip of the probe and the window surface (i.e. measurements 4 and 6), the results also show variations in levels of oxygenation and a higher degree of variance in general.

It is believed that the variations when no cap or a flexible cap was used are due to variations in spectroscope probe angle onto the skin surface cause a variation in specular reflections from the surface of the skin. The same is the case for the stiff caps with the extra gap between the probe tip and the window.

It can be seen that when the probe is brought into close proximity with the window portion (i.e. measurements 3 and 5) such that the gap is less than 0.50 mm (and potentially zero), the variance is significantly reduced.

Thus it will be appreciated by those skilled in the art that embodiments of the present invention provide a cap for use with probes such as DRS and microscope probes. The cap of the present invention may significantly reduce the ability for the probe angle to deviate from the desired angle (e.g. away from being normal to a patient's skin). Due to the rigidity of the cap, the cap does not flex when pushed around the subject's skin.

While specific embodiments of the invention have been described in detail, it will be appreciated by those skilled in the art that the embodiments described in detail are not limiting on the scope of the invention.

1. A system for measuring haemodynamic parameters relating to a subject, the system comprising:
a DRS probe;
a microscope probe; and
a cap for use at a respective distal end of each of the probes, one at a time, wherein the cap comprises:
a rigid flat contact surface for contact with a body surface of the subject, said contact surface comprising an aperture, said aperture being arranged such that, in use, the aperture is optically aligned with the optical probe in use;
a rigid side wall surrounding the contact surface, wherein the side wall defines a closed end and an open end, said closed end being formed by the contact surface and said open end being arranged to receive the probe in use, said side wall being arranged such that, in use, the cap is held in abutment against the probe; and
a securing portion arranged to removably secure the cap to each of the probes.

2. The system as claimed in claim 1, wherein the cap further comprises a substantially transparent window portion that covers the aperture of the contact surface.

3. The system as claimed in claim 2, wherein the cap is arranged such that, in use, a distance between the probe and the window portion is less than approximately 0.5 mm.

4. The system as claimed in claim 2, wherein the cap is arranged such that, in use, an operating end of the probe is in contact with the transparent window portion.

5. The system as claimed in claim 2, wherein the window portion has a thickness less than 1 mm, optionally between approximately 0.10 mm and 0.80 mm, further optionally between approximately 0.20 mm and 0.60 mm, further optionally between approximately 0.25 mm and 0.50 mm.

6. The system as claimed in claim 2, wherein the window portion comprises the same material as the contact surface and side wall, and optionally the securing portion.

7. The system as claimed in claim 2, wherein the window portion is affixed to the surface portion, optionally using glue or ultrasonic welding.

8. The system as claimed in claim 1, wherein an area of the contact surface is at least approximately 4 cm², optionally wherein the area of the contact surface is at least approximately 5 cm², further optionally wherein the area of the contact surface is at least approximately 6 cm².

9. The system as claimed in claim 1, wherein the contact surface and side wall are optically opaque.

10. The system as claimed in claim 1, wherein the cap is substantially cylindrical.

11. The system as claimed in claim 1, wherein the side wall comprises has a diameter less than a diameter of the securement portion.

12. The system as claimed in claim 1, wherein the side wall comprises has a diameter less than a diameter of the contact surface.

13. The system as claimed in claim 1, wherein the contact surface and the side wall comprise a polymer.

14. The system as claimed in claim 13, wherein the contact surface and the side wall comprise the same polymer.

15. The system as claimed in claim 1, wherein the securing portion comprises a polymer, optionally wherein the securing portion comprises the same polymer as the contact surface and/or side wall.

16. The system as claimed in claim 13, wherein the polymer is biocompatible.

17. The system as claimed in claim 13, wherein the polymer does not discolour when sterilised and/or stored.

18. The system as claimed in claim 1, wherein the contact surface and the side wall are integrally formed, optionally wherein the securement portion and the side wall are integrally formed.

19. The system as claimed in claim 1, wherein the securing portion comprises a plurality of protrusions extending from the side wall, wherein each of said protrusions comprises an engagement member arranged to engage with a corresponding engagement member on the probe.

20. The system as claimed in claim 19, wherein the engagement members of the cap comprise at least one of a tab, a rib, or a tongue; and/or wherein the corresponding engagement on the probe comprises a groove or a socket.

21. The system as claimed in claim 1, wherein the securement portion is less rigid than the contact surface and side wall.

22. An optical probe arrangement for measuring haemodynamic parameters relating to a subject, the system comprising:
an optical probe; and
a cap for use at said distal end of the probe, wherein the cap comprises:
a rigid flat contact surface for contact with a body surface of the subject, said contact surface comprising an aperture, said aperture being arranged such that, in use, the aperture is optically aligned with the optical probe;
a rigid side wall surrounding the contact surface, wherein the side wall defines a closed end and an open end, said closed end being formed by the contact surface and said open end being arranged to receive the probe, said side wall being arranged such that, in use, the cap is held in abutment against the probe; and
a securing portion arranged to removably secure the cap to the probe.

23. A cap for use at a distal end of an optical probe, wherein the cap comprises:
a rigid flat contact surface for contact with a body surface of a subject, said contact surface comprising an aperture, said aperture being arranged such that, in use, the aperture is optically aligned with the optical probe;
a rigid side wall surrounding the contact surface, wherein the side wall defines a closed end and an open end, said closed end being formed by the contact surface and said open end being arranged to receive the probe, said side wall being arranged such that, in use, the cap is held in abutment against the probe; and
a securing portion arranged to removably secure the cap to the probe.

24-27. (canceled)
